(19) United States
(12) Patent Application Publication
(10) Pub. No.: US 2026/0167541 A1
(43) Pub. Date: Jun. 18, 2026

(54) BIOCIDAL CONTROL IN AQUEOUS MEMBRANE SEPARATION SYSTEMS

(71) Applicant: Albemarle Corporation, Charlotte, NC (US)

(72) Inventor: Eric W. LIIMATTA, Baton Rouge, LA (US)

(73) Assignee: Albemarle Corporation, Charlotte, NC (US)

(21) Appl. No.: 19/510,539

(22) PCT Filed: Aug. 14, 2024
(86) PCT No.: PCT/US2024/042246

§ 371 Date: Feb. 11, 2026

Related U.S. Application Data
(60) Provisional application No. 63/532,796, filed on Aug. 15, 2023.

(51) Int. Cl. C02F 1/76; A01N 25/22; A01N 25/34; A01N 59/00; B01D 61/04

(52) U.S. Cl. CPC C02F 1/76; A01N 25/22; A01N 25/34; A01N 59/00; B01D 61/04

(57) ABSTRACT

This invention provides processes for microbial control in aqueous membrane separation systems, in which the microbial control is provided by bromine-containing biocides. The process for controlling biofouling in an aqueous membrane separation system comprising water and one or more protected membranes. The process comprises: I) contacting, upstream of the protected membrane(s), feed water and a biocidal amount of a bromine-containing biocide to form treated water; II) measuring a bromine residual in the treated water at a monitoring location which is downstream of the contacting in I) and upstream of the protected membrane(s); and III) contacting the treated water and a reducing amount of one or more reducing agents near or downstream of the monitoring location and upstream of the protected. membrane(s); wherein the reducing agent is capable of reducing biocidal bromine to bromide ions.

### RELATED APPLICATIONS

This application claims the benefit of U.S. Provisional Patent Application Ser. No. 63/532,796, filed Aug. 15, 2023, the disclosure of which is incorporated by reference herein in its entirety.

### TECHNICAL FIELD

This invention relates to biocidal control in aqueous membrane separation systems.

### BACKGROUND

Microbes, e.g., bacteria, fungi, and algae, entering an aqueous membrane separation system can deposit on the membrane and develop into biofilm, negatively affecting the performance of the membrane. The biofilm can impact the water passing through the membrane, block the membrane, and/or decrease the efficiency of the membrane.

Various methods for controlling or minimizing microbial growth and biofilm formation, especially on the membrane(s) of the system, have been developed. Some of these methods employ a biocide that contacts the membrane, which may damage the membrane. Methods that minimize or prevent biofilm formation on the membrane(s) without damaging the membranes are still desired.

### BRIEF DESCRIPTION OF THE DRAWINGS

The presently disclosed subject matter can be better understood by referring to the following, example FIGURE. The components in the FIGURE are not necessarily to scale, emphasis instead being placed upon illustrating the principles of the presently disclosed subject matter (often schematically). In the FIGURE, like reference numerals designate corresponding parts throughout the different views. A further understanding of the presently disclosed subject matter can be obtained by reference to an embodiment set forth in the illustrations of the accompanying drawing. Although the illustrated embodiment is merely for purposes of example of systems for carrying out the presently disclosed subject matter, both the organization and method of operation of the presently disclosed subject matter, in general, together with further objectives and advantages thereof, may be more easily understood by reference to the drawings and the following description. The drawing is not intended to limit the scope of this presently disclosed subject matter, which is set forth with particularity in the claims as appended or as subsequently amended, but merely to clarify and provide examples of the presently disclosed subject matter.

FIG. 1 is a graphical depiction of data from experiments conducted to test the progression of membrane degradation.

### SUMMARY OF THE INVENTION

This invention provides processes for control and minimization of microbial growth, especially biofilm growth in aqueous membrane separation systems. In the processes of this invention, biofouling is effectively minimized or controlled with little or no contact of the biocide with the membrane, which in turn, minimizes or prevents damage to the membrane by the biocide. By the practice of this invention, effective minimization of microbiological contamination of the water and the membrane(s) can be achieved.

An embodiment of this invention is a process for controlling biofouling in an aqueous membrane separation system comprising water and one or more protected membranes. The process comprises:
I) contacting, upstream of the protected membrane(s), the feed water and a biocidal amount of a bromine-containing biocide to form treated water;
II) measuring a bromine residual in the treated water at a monitoring location which is downstream of the contacting in I) and upstream of the protected membrane(s); and
III) contacting the treated water and a reducing amount of one or more reducing agents near or downstream of the monitoring location and upstream of the protected membrane(s).

The reducing agent in these processes is capable of reducing biocidal bromine to bromide ions. In these processes, the bromine-containing biocide comprises:
A) one or more 1,3-dibromo-5,5-dialkylhydantoins;
B) one or more N,N′-bromochloro-5,5-dialkylhydantoins;
C) one or more alkali metal hypobromites and/or one or more alkaline earth metal hypobromites, optionally formed in water from (i) one or more bromide sources and (ii) one or more hypochlorite salts and/or hypochlorous acid;
D) a bromine-based biocide formed in water from (i) one or more bromide sources, (ii) an oxidant, optionally (iii) at least one inorganic base, and optionally (iv) sulfamic acid and/or a metal salt of sulfamic acid;
E) a bromine-based biocide formed in water from (i) bromine chloride or bromine chloride and bromine, with or without conjoint use of chlorine, and (ii) overbased alkali metal salt of sulfamic acid and/or sulfamic acid, alkali metal base, and water, wherein (i) and (ii) are in relative proportions such that there is an atom ratio of nitrogen to active bromine greater than 0.93, and wherein the bromine-based biocide has a pH of greater than 7;
F) a bromine-based biocide formed in water by ozonation of one or more bromide sources selected from ammonium bromide, hydrogen bromide, one or more alkali metal bromides, one or more alkaline earth metal bromides, and mixtures of any two or more of the foregoing; or
G) a bromine-based biocide formed in water electrolytically from one or more bromide sources selected from ammonium bromide, hydrogen bromide, one or more alkali metal bromides, one or more alkaline earth metal bromides, and mixtures of any two or more of the foregoing.

These and other embodiments and features of this invention will be still further apparent from the ensuing description and appended claims.

### FURTHER DETAILED DESCRIPTION OF THE INVENTION

As used throughout this document, the phrase “biocidal amount” denotes that the amount used controls, kills, or otherwise reduces the bacterial or microbial content of the water being treated by a statistically significant amount.

The term ppm means parts per million (wt/wt), unless specifically stated otherwise herein.

An “aqueous membrane separation system” is sometimes referred to as an “aqueous membrane system” or as a “membrane system” throughout this document.

Throughout this document, the phrase “protected membrane” refers to a membrane in the aqueous membrane separation system which does not contact the biocide or has minimal contact with the biocide. Similarly, as used throughout this document, the phrase “non-protected membrane” refers to a membrane which contacts, or is permitted to contact, a biocide in the practice of this invention.

As used throughout this document, “downstream” means in the direction of travel of the feed water and treated water, and “upstream” means against (opposite to) the direction of travel of the feed water and treated water. In an aqueous membrane separation system, protected membranes are typically downstream from the feed water, and non-protected membranes are generally upstream of protected membranes.

In the processes of this invention, a biocidal amount of a bromine-containing biocide and feed water are contacted to form treated water; bromine-containing biocides in the treated water are measured as bromine residual at a monitoring location which is downstream of the contacting of the feed water and the bromine-containing biocide and upstream of the protected membrane(s); and the treated water and one or more reducing agents are contacted near or downstream of the monitoring location and upstream of the protected membrane(s). The bromine-containing biocide in the processes of this invention comprises bromine-containing biocides A), B), C), D), E), or F) as described above. The reducing agent in the processes of this invention is capable of reducing biocidal bromine to bromide ions.

Aqueous membrane separation system are aqueous systems containing one or more membranes in which one more membranes are used as part of a separation process. In some aqueous membrane separation systems, there is one membrane, which is a protected membrane. In other aqueous membrane separation systems, there are two or more membranes, and at least one of these membranes is a protected membrane. When there are two or more membranes in the aqueous membrane separation system, there may be both at least one protected membrane and at least one non-protected membrane.

Various aqueous membrane separation systems can be treated pursuant to this invention. Non-limiting examples of aqueous membrane separation systems that can be treated include a microfiltration system, an ultrafiltration system, a nanofiltration system, a reverse osmosis system, and/or an electrodialysis system. Reverse osmosis systems are preferred aqueous membrane separation systems for the processes of this invention.

In some embodiments, when the aqueous membrane separation system is a reverse osmosis (RO) system with a membrane upstream of the RO membrane, the upstream membrane may be a non-protected membrane, while the RO membrane is a protected membrane. In other embodiments, both the upstream membrane and the RO membrane are protected membranes.

As used throughout this document, the phrase “feed water” refers to the water for the aqueous membrane separation system prior to the contact of the water and the bromine-containing biocide; feed water includes water that will be fed to the aqueous membrane separation system. The phrase “treated water,” as used throughout this document, refers to (feed) water that has been contacted with a bromine-containing biocide pursuant to this invention.

The feed water can be from any convenient source, including desalinization water, industrial water, river water, marsh water, brackish surface and groundwater, and seawater.

Contacting of the bromine-containing biocide and the feed water is usually accomplished by introducing the bromine-containing biocide into the feed water. Bromine-containing biocides A) and B) are solids, and can be contacted with the feed water in solid form and/or the solids can be dissolved in water and then contacted with the feed water. Preferably, bromine-containing biocides A) and B) are dissolved in water and then contacted with the feed water. Bromine-containing biocides C), D), E), F), and G) are aqueous solutions and can be diluted with water prior to contact with the feed water to form diluted bromine-based biocides of C), D), E), F), and G), or the bromine-based biocides of C), D), E), F), and G) can be used without dilution for the contacting with the feed water.

The contacting of the bromine-containing biocide and the feed water can occur anywhere upstream of the protected membrane(s). Preferably, the bromine-containing biocide is contacted with the feed water as far upstream from the protected membrane(s) as possible. Early contact of the bromine-containing biocide and the feed water allows for longer contact time of the bromine-containing biocide and the feed water to obtain the most benefit from the bromine-containing biocide. Due to the presence of the bromine-containing biocide, the treated water has a more positive potential than the potential of the feed water.

In some embodiments, the feed water is introduced to the aqueous membrane separation system from a vessel such as a storage tank. In embodiments in which the feed water is contained in, or passes through, a vessel, contacting of the bromine-containing biocide and the feed water preferably occurs in the line transporting the feed water into the vessel.

The amount of bromine-containing biocide used depends on the demand in the feed water and the amount of microbial load present in the feed water or in contact with the feed water (e.g., biofilm). Preferably, the amount of bromine-containing biocide used is sufficient to control or meet the microbial load, or more preferably, the amount of bromine-containing biocide meets the microbial load and provides a bromine residual in the treated water. When the amount of bromine-containing biocide provides a bromine residual in the treated water, the bromine residual in the treated water is about 0.2 ppm to about 20 ppm, preferably about 0.5 ppm to about 10 ppm, more preferably about 0.5 ppm to about 2 ppm (wt/wt) as free bromine. When the feed water contacts the bromine-containing biocide, the feed water becomes treated water.

When the amount of bromine-containing biocide brought into contact with the feed water is not effective for biocidal control, additional bromine-containing biocide can be brought into contact with the treated water at any point upstream of the monitoring location. Preferably, the additional bromine-containing biocide is brought into contact with the treated water as far upstream of the monitoring location as possible. The additional bromine-containing biocide can be the same or different than the bromine-containing biocide initially brought into contact with the feed water.

The term “active bromine” refers to all of the bromine-containing species that are capable of biocidal activity. It is generally accepted all of the bromine in the +1 oxidation state is biocidally active and is thus included in the term “active bromine”. As is well known in the art, bromine, bromine chloride, hypobromous acid, hypobromite ion (OBr⁻), hydrogen tribromide, tribromide ion, and organo-N-brominated compounds have bromine in the +1 oxidation state. Thus these, as well as other species with bromine in the +1 oxidation state, to the extent they are present, constitute the active bromine content of the bromine-containing biocides used in the practice of this invention.

For the measuring of a bromine residual in the treated water, the monitoring location is downstream of the contacting of the bromine-containing biocides and the feed water and upstream of the protected membrane(s). Preferably, the monitoring location is at a distance upstream of the protected membrane such that the reducing agent has time to contact the bromine-containing biocide.

In some embodiments, the measuring of a bromine residual in the treated water at the monitoring location includes chemical methods such as the N,N′-diethyldiphenylenediamine (DPD) test and spectroscopic methods such as UV/visible spectroscopy.

The presence of the bromine-containing biocide provides an oxidizing potential in the treated water. In some embodiments, this potential is measured and related to the amount of bromine-containing biocide in the treated water. Two methods for monitoring the potential of the treated water are preferred in the practice of this invention, oxidation-reduction potential and amperometry. Both methods involve placing a probe in the treated water and measuring the potential of the water, which provides an indication of the concentration of the bromine-based biocide at the monitoring location. Both amperometry and oxidation-reduction potential monitoring can be set up for intermittent or continuous measurement of the potential of the treated water. ORP sensors and controllers are commercially available, and amperometer electrodes and controllers are also commercially available.

Oxidation-reduction potential (ORP) measures the potential of the treated water, typically in millivolts; positive values indicate an oxidizing environment, and an increase in the ORP value from the ORP value of the feed water indicates that a bromine-containing biocide is present; negative values indicate a reducing environment, meaning that sufficient reducing agent has been contacted with the treated water to react with (“neutralize”) all of the bromine-containing biocide in the treated water.

In some embodiments, a desired ORP value after contact of the treated water and the reducing agent is about +250 mV to about −5 mV.

In the practice of this invention, the ORP value of the treated water is measured at the monitoring location, and the desired amount of reducing agent is contacted with the treated water. Preferably, sufficient reducing agent is contacted with the treated water to make the treated water nonoxidizing.

In some embodiments, the ORP can be connected to a controller for a reducing agent feed, and once the ORP value in the treated water reaches a preset value, the ORP can communicate with the reducing agent controller to stop introduction of the reducing agent.

Amperometry measures changes in current when applying a fixed potential to the treated water; positive values indicate an oxidizing environment, meaning that a bromine-containing biocide is present; negative values indicate a reducing environment, meaning that sufficient reducing agent has been contacted with the treated water to react with (“neutralize”) all of the bromine-containing biocide in the treated water.

In the practice of this invention, the amperometric potential value of the treated water is measured at the monitoring location, and the desired amount of reducing agent is contacted with the treated water. Preferably, sufficient reducing agent is contacted with the treated water to make the treated water nonoxidizing.

In some embodiments, the amperometer can be connected to a controller for a reducing agent feed, and once the potential measured in the treated water reaches a preset value, the amperometer can communicate with the reducing agent controller to stop introduction of the reducing agent.

The contacting of the reducing agent and the treated water is at or downstream of the monitoring location while also being upstream of the protected membrane, or upstream of the first protected membrane when the aqueous membrane separation system has more than one protected membrane.

In some embodiments, there is more than one monitoring location, and the second monitoring location is downstream of the first monitoring location; the monitoring method at each monitoring location may be the same or different. Additional reducing agent can be introduced to the water of the aqueous membrane separation system near or downstream of the second monitoring location and upstream of the protected membrane(s) as needed or desired.

In the practice of this invention, the reducing agents can be blended directly into the treated water. If desired, the reducing agent(s) can be pre-mixed with water prior to introduction into the treated water.

The amount of reducing agent used usually depends on the amount of bromine-containing biocide in the treated water. The reducing agent reduces the amount of bromine-containing biocide that contacts the protected membrane by consuming at least a portion of the bromine-containing biocide. As a result of the measuring of the bromine residual in the treated water, the amount of reducing agent that will contact the treated water can be adjusted, manually or automatically, to consume a specific amount of, or preferably, all of the bromine-containing biocide in the treated water. Preferably, the amount of reducing agent used is sufficient to minimize the amount of bromine residual in the treated water, or more preferably, the amount of reducing agent is sufficient to consume all of the bromine-containing biocide in the treated water.

When the amount of reducing agent brought into contact with the treated water is not effective for reducing the bromine residual in the treated water to the desired value, additional reducing agent can be brought into contact with the treated water at any point downstream of the monitoring location and upstream of the protected membrane(s). The additional reducing agent can be the same or different than the reducing agent initially brought into contact with the treated water.

The contacting of the treated water and the reducing agent forms reduced or neutralized water that can contact and pass through the protected membrane(s) with minimal or no damage to the protected membrane(s). Here, the term “neutralized” indicates that the reducing agent has been added in a sufficient amount to quench or neutralize the oxidizing power of the bromine-containing biocide. When sufficient reducing agent to consume all of the bromine-containing biocide is used, contact of the bromine-containing biocide and the protected membrane is prevented.

The bromine-containing biocides used pursuant to this invention are oxidizing biocides.

The 1,3-dibromo-5,5-dialkylhydantoins and N,N′-bromochloro-5,5-dialkylhydantoins used pursuant to this invention are solids, and can be blended directly into the feed water. Preferably, the 1,3-dibromo-5,5-dialkylhydantoin(s) and N,N′-bromochloro-5,5-dialkylhydantoin(s) are pre-mixed with water prior to contact with the feed water. In the pre-mix water (usually a solution), the microbiocidal amount of one or more 1,3-dibromo-5,5-dialkylhydantoins or one or more N,N′-bromochloro-5,5-dialkylhydantoins is preferably in an amount sufficient to provide a bromine residual in a range of about 300 ppm to about 3500 ppm, more preferably about 500 ppm to about 1200 ppm, still more preferably about 800 ppm to about 1000 ppm (wt/wt) as free bromine.

In the practice of this invention, the one or more 1,3-dibromo-5,5-dialkylhydantoins have alkyl groups containing one to about 4 carbon atoms. Preferred are 1,3-dibromo-5,5-dialkylhydantoins in which one of the alkyl groups is a methyl group and the other alkyl group contains in the range of 1 to about 4 carbon atoms. Thus, preferred 1,3-dibromo-5,5-dialkylhydantoins include 1,3-dibromo-5,5-dimethylhydantoin, 1,3-dibromo-5-ethyl-5-methylhydantoin, 1,3-dibromo-5-n-propyl-5-methylhydantoin, 1,3-dibromo-5-isopropyl-5-methylhydantoin, 1,3-dibromo-5-n-butyl-5-methylhydantoin, 1,3-dibromo-5-isobutyl-5-methylhydantoin, 1,3-dibromo-5-sec-butyl-5-methylhydantoin, 1,3-dibromo-5-tert-butyl-5-methylhydantoin, and mixtures of any two or more of them. Of these biocidal agents, 1,3-dibromo-5-isobutyl-5-methylhydantoin, 1,3-dibromo-5-n-propyl-5-methylhydantoin, and 1,3-dibromo-5-ethyl-5-methylhydantoin are preferred from a cost effectiveness standpoint. For mixtures of the foregoing 1,3-dibromo-5,5-dialkylhydantoins, it is preferred to use 1,3-dibromo-5,5-dimethylhydantoin as one of the components, with a mixture of 1,3-dibromo-5,5-dimethylhydantoin and 1,3-dibromo-5-ethyl-5-methylhydantoin being more preferred. A particularly preferred 1,3-dibromo-5,5-dialkylhydantoin is 1,3-dibromo-5,5-dimethylhydantoin.

Methods for producing 1,3-dibromo-5,5-dialkylhydantoins are known and reported in the literature, and some of them are available commercially.

The one or more N,N′-bromochloro-5,5-dialkylhydantoins used in the practice of this invention are N,N′-bromochloro-5,5-dialkylhydantoins in which each alkyl group independently contains in the range of 1 to about 4 carbon atoms. Suitable compounds of this type include, for example, such compounds as N,N′-bromochloro-5,5-dimethylhydantoin, N,N′-bromochloro-5-ethyl-5-methylhydantoin, N,N′-bromochloro-5-propyl-5-methylhydantoin, N,N′-bromochloro-5-isopropyl-5-methylhydantoin, N,N′-bromochloro-5-butyl-5-methylhydantoin, N,N′-bromochloro-5-isobutyl-5-methylhydantoin, N,N′-bromochloro-5-sec-butyl-5-methylhydantoin, N,N′-bromochloro-5-tert-butyl-5-methylhydantoin, N,N′-bromochloro-5,5-diethylhydantoin, and mixtures of any two or more of the foregoing. Most preferred is N,N′-bromochloro-5,5-dimethylhydantoin.

When a mixture of two or more N,N′-bromochloro-5,5-dialkylhydantoin biocides is used pursuant to this invention, the individual biocides of the mixture can be in any proportions relative to each other. Minor proportions (less than 50 wt %) of mono-N-bromo-5,5-dialkylhydantoin(s) can also be present, either with such mixtures of two or more N,N′-bromochloro-5,5-dialkylhydantoin biocides, or with only one N,N′-bromochloro-5,5-dialkylhydantoin biocide. One suitable mixture has a predominate amount by weight of N,N′-bromochloro-5,5-dimethylhydantoin together with a minor proportion by weight of 1,3-dichloro-5,5-dimethylhydantoin and 1,3-dichloro-5-ethyl-5-methylhydantoin.

Methods for producing such N,N′-bromochloro-5,5-dialkylhydantoins are known and reported in the literature, and some of them are available commercially. For example, N,N′-bromochloro-5,5-dimethylhydantoin is available commercially under the trade designation BromiCide® biocide (BWA Water Additives UK Limited).

Biocide C) is one or more alkali metal hypobromites and/or one or more alkaline earth metal hypobromites, preferably formed in water from (i) one or more bromide sources and (ii) a hypochlorite salt and/or hypochlorous acid.

In the practice of this invention, the bromide source and the hypochlorite salt and/or hypochlorous acid can contact the feed water separately (e.g., by separate inlets into the feed water); or the bromide source and the hypochlorite salt and/or hypochlorous acid can be fed to a mixing point (e.g., a mixing tee) or mixing device (preferably an in-line mixing device) and then contacted with the feed water; or the bromide source and the hypochlorite salt and/or hypochlorous acid can be combined in water to form an aqueous solution which is brought into contact with the feed water.

For forming bromine-containing biocides C), suitable bromide sources for ingredient (i) include ammonium bromide, hydrogen bromide, inorganic bromide salts, and mixtures of any two or more of the foregoing. The inorganic bromide salts are preferably one or more alkali metal bromides and/or one or more alkaline earth metal bromides. Preferred bromide sources are ammonium bromide, hydrogen bromide, alkali metal bromides including lithium bromide, sodium bromide, potassium bromide, and alkaline earth metal bromides, particularly MgBr₂ and CaBr₂. Mixtures of two or more bromide sources can be used if desired. A preferred bromide source is sodium bromide, especially sodium bromide from which trace amounts of alcohol such as methanol have been removed.

In formation of biocide C), a hypochlorite salt is preferred, and the hypochlorite salt is an inorganic hypochlorite salt, preferably one or more alkali metal hypochlorites and/or one or more alkaline earth metal hypochlorites. The alkali metal hypochlorites and alkaline earth metal hypochlorites include lithium hypochlorite, sodium hypochlorite, potassium hypochlorite, calcium hypochlorite, magnesium hypochlorite, and the like; sodium hypochlorite and calcium hypochlorite are preferred hypochlorite salts. Bromides or hypochlorites of Be, Sr, or Ba are not recommended because of toxicological concerns. Thus, the term “alkaline earth” as used herein excludes Be, Sr, and Ba. When ammonium bromide is the bromide source, it is desirable to mix the ammonium bromide with the hypochlorite salt in the manner described in U.S. Pat. No. 6,478,973.

The interaction of the bromide source and the hypochlorite salt and/or hypochlorous acid results in an aqueous solution having a suitably high bromine residual. If an excess amount of the hypochlorite is used relative to the amount of bromide salt used, the resultant solution will contain chlorine-based species as well as a bromine residual. These chlorine-based species are not harmful as long as the desired bromine residual is present in the solution being used.

In some embodiments, the one or more bromide sources and the one or more hypochlorite salt and/or hypochlorous acid used in the formation of biocide C) are contacted with the feed water separately, and the alkali metal hypobromites and/or one or more alkaline earth metal hypobromites are formed in the feed water. In these embodiments, the bromide sources, which are typically solids, and can be contacted with the feed water in solid form and/or, preferably, dissolved in water to form an aqueous solution and then contacted with the feed water. In these embodiments, the hypochlorite salts and/or hypochlorous acid are usually and preferably contacted with the feed water as aqueous solutions, although hypochlorite salts can be contacted with the feed water in solid form if desired.

In other embodiments, the one or more bromide sources and the one or more hypochlorite salt and/or hypochlorous acid used in the formation of biocide C) are combined before contacting the feed water. In these embodiments, the bromide sources are preferably dissolved in water to form an aqueous solution before being combined with the hypochlorite salts and/or hypochlorous acid. In these embodiments, the hypochlorite salts and/or hypochlorous acid are usually and preferably in the form of aqueous solutions when combined with the bromide sources.

When the bromide source and the hypochlorite salt and/or hypochlorous acid are combined in water to form an aqueous solution, the aqueous solution can be diluted with water prior to contact with the feed water to form a diluted bromine-containing biocide C) solution, or the bromine-containing biocide C) solution can be used without dilution for the contacting with the feed water.

Bromine-based biocides of D) and E) are aqueous solutions, and contain active bromine, also referred to as a bromine residual. The bromine-based biocides of D) and E) that contain sulfamic acid and/or a metal salt of sulfamic acid are stabilized bromine-based biocides.

Bromine-based biocides of D) are formed in water from (i) one or more bromide sources, (ii) an oxidant, optionally (iii) at least one inorganic base, and optionally (iv) sulfamic acid and/or a metal salt of sulfamic acid. This bromine-based biocide is preferably made as a concentrated aqueous solution; preferably, the concentrated solution is contacted with the feed water.

When an inorganic base is used, the pH is normally about 7 or greater and preferably is higher than 7, e.g., a pH in the range of about 10 to about 14.

For forming bromine-based biocides D), suitable bromide sources for ingredient (i) include ammonium bromide, hydrogen bromide, inorganic bromide salts, and mixtures of any two or more of the foregoing. The inorganic bromide salts are preferably one or more alkali metal bromides and/or one or more alkaline earth metal bromides. Preferred bromide sources are ammonium bromide, hydrogen bromide, alkali metal bromides including lithium bromide, sodium bromide, potassium bromide, and alkaline earth metal bromides, particularly MgBr₂ and CaBr₂. Mixtures of two or more bromide sources can be used if desired. A preferred bromide source is sodium bromide, especially sodium bromide from which trace amounts of alcohol such as methanol have been removed.

Oxidants include chlorine oxidants, and oxygen-based oxidants. Chlorine oxidants include elemental chlorine (Cl₂), hypochlorite salts, trichloroisocyanurate, and sodium dichloroisocyanurate. When the chlorine oxidant is hypochlorous acid or a hypochlorite salt, an inorganic base is usually present; preferably, sulfamic acid and/or a metal sulfamate salt is also present. Oxygen-based oxidants include ozone, peracetic acid, and hydrogen peroxide. When the oxygen-based oxidant is ozone, an inorganic base and/or sulfamic acid (and/or a metal salt of sulfamic acid) is usually present; preferably, both an inorganic base and sulfamic acid (and/or a metal salt of sulfamic acid) are present.

In some embodiments of bromine-based biocide D), an inorganic base is present. Preferred inorganic bases are alkali metal bases, more preferred are an oxide or hydroxide of lithium, sodium, and/or potassium, even more preferred are sodium hydroxide and/or potassium hydroxide.

Sulfamic acid and/or a metal salt of sulfamic acid is optional but preferred in some bromine-based biocides D). Metal salts of sulfamic acid are usually the alkali metal salts, including lithium sulfamate, sodium sulfamate, and potassium sulfamate. Sulfamic acid can be used alone or in a mixture with one or more metal salts of sulfamic acid. Sulfamic acid and/or sodium sulfamate are preferred. Sulfamic acid is more preferred. An inorganic base is optional but preferred when sulfamic acid is used to form bromine-based biocides D). In some embodiments of bromine-based biocide D), sulfamic acid and/or a metal salt of sulfamic acid is preferred.

In some preferred embodiments of bromine-based biocide D), the oxidant is trichloroisocyanurate or sodium dichloroisocyanurate, and an inorganic base is present.

If an excess amount of a chlorine oxidant is used relative to the amount of bromide salt used, the resultant solution will contain chlorine-based species as well as a bromine residual. These chlorine-based species are not harmful as long as the desired bromine residual is present in the solution being used.

A commercial aqueous bromine-based biocide D) that can be utilized in practicing this invention is available under the trade designation Sta·Br·Ex® biocide (Ecolab USA Inc.). This product contains active bromine stabilized against chemical decomposition and physical evaporation of active bromine species by the inclusion of sulfamate. For additional details concerning preparation of aqueous biocidal solutions stabilized with sulfamic acid, see U.S. Pat. Nos. 6,007,726; 6,156,229; and 6,270,722.

Another bromine-based biocide D) is available commercially under the trade designation BromMax® biocide (Enviro Tech Chemical Services, Inc.). This product contains active bromine stabilized against chemical decomposition and physical evaporation of active bromine species by the inclusion of sulfamate. For additional details concerning preparation of this type of bromine-based biocide D) stabilized with sulfamic acid, see U.S. Pat. Nos. 7,045,153; 7,309,503; and 7,455,859.

Still another commercial bromine-based biocide D) that can be utilized in practicing this invention is available under the trade designation Justeq07 biocide (Justeq, LLC). This product contains active halogen species stabilized by the inclusion of sulfamate. In some embodiments, this biocide may be made by producing chlorosulfamate from a chlorine oxidant, preferably a hypochlorite salt, and sulfamic acid and/or a metal salt of sulfamic acid, and then introducing the bromide source. Processes for producing aqueous biocide solutions of this type are described in U.S. Pat. Nos. 6,478,972; 6,533,958; and 7,341,671.

Bromine-based biocides of E) are formed in water from (i) bromine chloride or bromine chloride and bromine, with or without conjoint use of chlorine, and (ii) overbased alkali metal salt of sulfamic acid and/or sulfamic acid, alkali metal base, and water, wherein (i) and (ii) are in relative proportions such that there is an atom ratio of nitrogen to active bromine greater than 0.93, and wherein the bromine-based biocide has a pH of greater than 7. Bromine-based biocide D) is preferably made as a concentrated aqueous solution; preferably, the concentrated solution is contacted with the feed water.

Processes for producing aqueous bromine-based biocides E) are described in U.S. Pat. Nos. 6,068,861 and 6,299,909 B1. Bromine-based biocides E) containing over 50,000 ppm of active halogen is available commercially from Albemarle Corporation under the trademark STABROM® 909 biocide (Albemarle Corporation); the pH of the aqueous product as received is normally in the range of 13 to 14.

When forming bromine-based biocide E), the pH is normally at least 7 and preferably is always at a pH higher than 7, e.g., in the range of 10-14, by use of an inorganic base. Preferred bases are alkali metal bases, preferably an oxide or hydroxide of lithium, sodium, and/or potassium, more preferably sodium hydroxide and/or potassium hydroxide. If sulfamic acid is used in forming concentrated aqueous biocidal solution, the solution should also be provided with a base, preferably sufficient base to keep the solution alkaline, i.e., with a pH above 7, preferably about 10 or above, and most preferably about 13 or above.

For ingredient (i) of bromine-based biocide E), bromine chloride, a mixture of bromine chloride and bromine, or a combination of bromine and chlorine in which the molar amount of chlorine is either equivalent to the molar amount of bromine or less than the molar amount of bromine is used, the aqueous biocide solution is bromine-based as most of the chlorine usually forms chloride salts such as sodium chloride since an alkali metal base such as sodium hydroxide is typically used in the processing to raise the pH of the product solution to about 13 or greater.

When a concentrated aqueous solution of bromine-based biocide E) is made, the active bromine content of such aqueous biocide solutions is usually about 50,000 ppm (wt/wt) or more; preferably, about 100,000 ppm (wt/wt) or more, e.g., as much as about 105,000 to about 215,000 ppm of active bromine. The pH of such concentrated aqueous biocide solutions is greater than 7, preferably about 10 or greater, more desirably about 12 or greater, and still more desirably about 13 or greater, and the atom ratio of nitrogen to active bromine in these separate aqueous biocide solutions is greater than 0.93.

Bromine-based biocide F) is formed in water by ozonation of one or more bromide sources. This bromine-based biocide is made by ozonating an aqueous solution containing bromide ions by passing the solution containing bromide ions through an ozonation device. The ozonated solution contains biocidally active bromine, and is normally contacted with the feed water a short time after ozonation, preferably by transporting the solution directly from the ozonation process into contact with the feed water.

For forming bromine-based biocides F), suitable bromide sources include ammonium bromide, hydrogen bromide, inorganic bromide salts, and mixtures of any two or more of the foregoing. The inorganic bromide salts are preferably one or more alkali metal bromides and/or one or more alkaline earth metal bromides. Preferred bromide sources are ammonium bromide, hydrogen bromide, alkali metal bromides including lithium bromide, sodium bromide, potassium bromide, and alkaline earth metal bromides, particularly MgBr₂ and CaBr₂. Mixtures of two or more bromide sources can be used if desired. A preferred bromide source is sodium bromide, especially sodium bromide from which trace amounts of alcohol such as methanol have been removed.

Bromine-based biocide G) is formed in water electrolytically from one or more bromide sources. This bromine-based biocide is made by electrolytically generating bromine in an aqueous solution containing bromide ions, typically by passing the solution containing bromide ions through an electrogeneration system. The electrolyzed solution contains biocidally active bromine, and is normally contacted with the feed water a short time after electrolysis, preferably by transporting the solution directly from the electrolysis process into contact with the feed water.

For forming bromine-based biocides G), suitable bromide sources include ammonium bromide, hydrogen bromide, inorganic bromide salts, and mixtures of any two or more of the foregoing. The inorganic bromide salts are preferably one or more alkali metal bromides and/or one or more alkaline earth metal bromides. Preferred bromide sources are ammonium bromide, hydrogen bromide, alkali metal bromides including lithium bromide, sodium bromide, potassium bromide, and alkaline earth metal bromides, particularly MgBr₂ and CaBr₂. Mixtures of two or more bromide sources can be used if desired. A preferred bromide source is sodium bromide, especially sodium bromide from which trace amounts of alcohol such as methanol have been removed.

Of the several types of bromine-containing biocides that can be used in the practice of this invention, preferred bromine-containing biocides include N,N′-bromochloro-5,5-dialkylhydantoins, 1,3-dibromo-5,5-dialkylhydantoins, and bromine-based biocides formed in water from bromine chloride or bromine chloride and bromine. More preferred bromine-containing biocides include N,N′-bromochloro-5,5-dimethylhydantoin, 1,3-dibromo-5,5-dimethylhydantoin, and bromine-based biocides formed in water from bromine chloride; particularly preferred are bromine-based biocides formed in water from bromine chloride.

The desired bromine residual in a bromine-containing biocide solution before contacting with the feed water can depend on a variety of factors such as known microbial demand downstream of the location of the contacting of the feed water and the bromine-containing biocide. In some embodiments, the bromine residual in the feed water after contacting a bromine-containing biocide is about 0.2 ppm to about 20 ppm, preferably about 0.5 ppm to about 10 ppm, more preferably about 0.5 ppm to about 2 ppm (wt/wt) as free bromine.

This invention permits use of bromine-containing biocides and one or more other microbiocidal agents that are compatible therewith. Preferably, the bromine-containing biocides are the sole sources of microbiocidal activity in the water pursuant to this invention.

The reducing agents used pursuant to this invention are capable of reducing biocidal bromine to bromide ions (Br⁻). Reducing agents that can be used in the practice of this invention are any reducing agent that is effective at reducing a bromine-based oxidant, or a reducing agent that is effective at reducing the particular bromine-based oxidant(s) present in the membrane separation system. Typical reducing agents in the practice of this invention include sulfur dioxide, sodium metabisulfite, sodium hydrogen sulfite, sodium bisulfite, sodium sulfite, sodium bisulfate, sodium thiosulfate, ammonium bisulfite, and ammonium thiosulfate. Mixtures of any two or more reducing agents can be used. In some embodiments, it is convenient to use one reducing agent rather than a mixture of reducing agents.

The term “free bromine” is used to describe the free or relatively fast-reacting forms of bromine oxidants present in aqueous solutions. In the case of the bromine-containing biocides used in the practice of this invention, total bromine is the same as active bromine. To convert “free chlorine” and “total chlorine” values (e.g., ppm Cl₂) into “free bromine” and “total bromine” values (e.g., ppm Br₂), the given concentration for “free chlorine” or “total chlorine” in terms of ppm Cl₂ is multiplied by 2.25, the molecular weight ratio of Br₂ to Cl₂. Similarly, when the given concentration of halogen is reported as Br₂, it can be converted to a Cl₂ value by dividing by 2.25, the molecular weight ratio of Cl₂ to Br₂.

The term “bromine residual” refers to the amount (concentration) of bromine species present in the water and available for disinfection. Residuals can be determined as either “free” or “total” depending upon the analytical test method employed. In the present case, the numerical values for bromine residual have been given herein on a free bromine basis. Such values can be monitored by use of the analytical procedure for “free chlorine” given below. However if desired, the bromine residual could be monitored on a “total bromine” basis by using the analytical procedure for “total chlorine” given below. In either case the numerical values obtained are in terms of chlorine and thus such values are multiplied by 2.25 to obtain the corresponding bromine values. Typically the values on a “total bromine” basis on a given sample will be higher than the values on a “free bromine” basis on the same given sample. The important point to understand is that this invention relates to the bromine residual that is actually present in the treated aqueous medium whether the value is determined by use of the free chlorine test procedure or the total chlorine test procedure, but use of the free chlorine test procedure is recommended.

Suitable methods for determining “bromine residual” are known and reported in the literature. See for example, Standard Methods For the Examination of Water and Wastewater, 18th Edition, 1992, from American Public Health Association, 1015 Fifteenth Street, NW, Washington, DC 20005 (ISBN 0-87553-207-1), pages 4-36 and 4-37; Hach Water Analysis Handbook, Third Edition, 1997, by Hach Company, Loveland Colorado, especially pages 1206 and 1207; and Handbook of Industrial Water Conditioning, 7th edition, Betz Laboratories, Inc., Trevose, PA 19047 (Library of Congress Catalog Card Number: 76-27257), 1976, pages 24-29. While these references typically refer to “chlorine residual”, the same techniques are used for determining “bromine residual”, by taking into account the higher atomic weight of bromine as compared to chlorine.

Active halogen content, whether active chlorine, active bromine, or both, is determinable by use of conventional starch-iodine titration.

A standard test for determination of low levels of active halogen is known as the DPD test and is based on classical test procedures devised by Palin in 1974. See A. T. Palin, “Analytical Control of Water Disinfection With Special Reference to Differential DPD Methods For Chlorine, Chlorine Dioxide, Bromine, Iodine and Ozone”, J. Inst. Water Eng., 1974, 28, 139. While there are various modernized versions of the Palin procedures, the recommended version of the test is fully described in Hach Water Analysis Handbook, 3rd edition, copyright 1997. The procedure for “total chlorine” (i.e., active chlorine) is identified in that publication as Method 8167 appearing on page 379, Briefly, the “total chlorine” test involves introducing to the dilute water sample containing active halogen, a powder comprising DPD indicator powder, (i.e., N,N′-diethyldiphenylenediamine, KI, and a buffer). The active halogen species present react(s) with KI to yield iodine species which turn the DPD indicator to red/pink. The intensity of the coloration depends upon the concentration of “total chlorine” species (i.e., active chlorine”) present in the sample. This intensity is measured by a colorimeter calibrated to transform the intensity reading into a “total chlorine” value in terms of mg/L Cl₂. If the active halogen present is active bromine, the result in terms of mg/L Cl₂ is multiplied by 2.25 to express the result in terms of mg/L Br₂ of active bromine.

In greater detail, the DPD test procedure is as follows:
1. To determine the amount of species present in the water which respond to the “total chlorine” test, the water sample should be analyzed within a few minutes of being taken, and preferably immediately upon being taken.
2. Hach Method 8167 for testing the amount of species present in the water sample which respond to the “total chlorine” test involves use of the Hach Model DR 2010 colorimeter. The stored program number for chlorine determinations is recalled by keying in “80” on the keyboard, followed by setting the absorbance wavelength to 530 nm by rotating the dial on the side of the instrument. Two identical sample cells are filled to the 25 mL mark with the water under investigation. One of the cells is arbitrarily chosen to be the blank. To the second cell, the contents of a DPD Total Chlorine Powder Pillow are added. This is shaken for 10-20 seconds to mix, as the development of a pink-red color indicates the presence of species in the water which respond positively to the DPD “total chlorine” test reagent. On the keypad, the SHIFT TIMER keys are depressed to commence a three minute reaction time. After three minutes the instrument beeps to signal the reaction is complete. The blank sample cell is admitted to the sample compartment of the Hach Model DR 2010, and the shield is closed to prevent stray light effects. Then the ZERO key is depressed. After a few seconds, the display registers 0.00 mg/L Cl₂. Then, the blank sample cell used to zero the instrument is removed from the cell compartment of the Hach Model DR 2010 and replaced with the test sample to which the DPD “total chlorine” test reagent was added. The light shield is then closed as was done for the blank, and the READ key is depressed. The result, in mg/L Cl₂, is shown on the display within a few seconds. This is the “total chlorine” level of the water sample under investigation. It is to be noted that the test sample may need to be diluted with halogen demand free water in order for the chlorine measurement to be within the measuring range of the instrument. This dilution will need to be taken into account to determine the actual chlorine level of the sample.
3. One method for measuring free chlorine is the Hach Method 8021. This tests for the amount of species present in the water sample which respond to the “free chlorine” test. This test involves the use of the Hach Model DR 2010 colorimeter. The stored program number for chlorine determinations is recalled by keying in “80” on the keyboard, followed by setting the absorbance wavelength to 530 nm by rotating the dial on the side of the instrument. Two identical sample cells are filled to the 25 mL mark with the water under investigation. One of the cells is arbitrarily chosen to be the blank. The blank sample cell is admitted to the sample compartment of the Hach Model DR 2010, and the shield is closed to prevent stray light effects. Then the ZERO key is depressed. After a few seconds, the display registers 0.00 mg/L Cl₂. Then, the blank sample cell used to zero the instrument is removed from the cell compartment of the Hach Model DR 2010. To the second cell, the contents of a DPD Free Chlorine Powder Pillow are added. This is shaken for 10-20 seconds to mix, as the development of a pink-red color indicates the presence of species in the water which respond positively to the DPD “free chlorine” test reagent. Immediately (within one minute of reagent addition) place the prepared sample into the cell holder. The light shield is then closed as was done for the blank, and the READ key is depressed. The result, in mg/L Cl₂, is shown on the display within a few seconds. This is the “free chlorine” level of the water sample under investigation. It is to be noted that the test sample may need to be diluted with halogen demand free water in order for the chlorine measurement to be within the measuring range of the instrument. The dilution will need to be taken into account when determining the chlorine level of the sample.

In some embodiments, the aqueous membrane separation system is a reverse osmosis system, the biocide is an N,N′-bromochloro-5,5-dialkylhydantoin or a 1,3-dibromo-5,5-dialkylhydantoin, preferably N,N′-bromochloro-5,5-dimethylhydantoin or 1,3-dibromo-5,5-dimethylhydantoin, more preferably 1,3-dibromo-5,5-dimethylhydantoin, and the biocide is preferably dissolved in water before contacting the feed water. In these embodiments, the measuring of the potential of the treated water is preferably performed with an oxidation reduction potential meter. When there is a bromine residual in the treated water, the bromine residual is about 0.2 ppm to about 20 ppm, preferably about 0.5 ppm to about 10 ppm, more preferably about 0.5 ppm to about 2 ppm (wt/wt) as free bromine.

In other embodiments, the aqueous membrane separation system is a reverse osmosis system, the biocide is a bromine-based biocide formed in water from bromine chloride or bromine chloride and bromine, more preferably, a bromine-based biocide formed in water from bromine chloride and the biocide is preferably contacted with the feed water without dilution. In these embodiments, the measuring of the potential of the treated water is preferably performed with an oxidation reduction potential meter. When there is a bromine residual in the treated water, the bromine residual is about 0.2 ppm to about 20 ppm, preferably about 0.5 ppm to about 10 ppm, more preferably about 0.5 ppm to about 2 ppm (wt/wt) as free bromine.

In still other embodiments, the aqueous membrane separation system is a reverse osmosis system, the biocide is an N,N′-bromochloro-5,5-dialkylhydantoin or a 1,3-dibromo-5,5-dialkylhydantoin, preferably N,N′-bromochloro-5,5-dimethylhydantoin or 1,3-dibromo-5,5-dimethylhydantoin, more preferably 1,3-dibromo-5,5-dimethylhydantoin, and the biocide is preferably contacted with the feed water in solid form. In these embodiments, the measuring of the potential of the treated water is preferably performed with an amperometer. When there is a bromine residual in the treated water, the bromine residual is about 0.2 ppm to about 20 ppm, preferably about 0.5 ppm to about 10 ppm, more preferably about 0.5 ppm to about 2 ppm (wt/wt) as free bromine.

In yet other embodiments, the aqueous membrane separation system is a reverse osmosis system, the biocide is a bromine-based biocide formed in water from bromine chloride or bromine chloride and bromine, more preferably, a bromine-based biocide formed in water from bromine chloride and the biocide is preferably contacted with the feed water without dilution. In these embodiments, the measuring of the potential of the treated water is preferably performed with an amperometer. When there is a bromine residual in the treated water, the bromine residual is about 0.2 ppm to about 20 ppm, preferably about 0.5 ppm to about 10 ppm, more preferably about 0.5 ppm to about 2 ppm (wt/wt) as free bromine.

The following examples are presented for purposes of illustration, and are not intended to impose limitations on the scope of this invention.

### Example 1

Marsh water was fed into a storage tank via a 1200-foot long (365 m) hose. A biocide was added to the hose at a point 20 feet (6.1 m) before the hose connection to the storage tank. Water was fed from the storage tank to a reverse osmosis (RO) system equipped with an ultrafiltration (UF) membrane upstream of an RO membrane. In this system, the UF membrane was a non-protected membrane and the RO membrane was a protected membrane. An oxidation-reduction potential (ORP) electrode was in the water at a point upstream of and near the RO membrane, and the ORP electrode was connected to an ORP controller unit (ORP electrode: RD2, Hach Company). Measurements from the ORP were monitored, and the reducing agent was pumped into the RO system downstream of the ORP electrode as needed as indicated by the ORP reading from the ORP electrode.

The biocides tested were bleach, 1,3-dibromo-5,5-dimethylhydantoin (Br₂DMH; Albemarle Corporation), and an aqueous solution formed from bromine chloride and sulfamic acid containing over 50,000 ppm of active halogen having a pH in the range of 13 to 14 (BrCl/sulfamic acid; STABROM® 909 biocide, Albemarle Corporation). The reducing agent used with each biocide was sodium bisulfate.

The water was sampled at various times upstream of and near the UF membrane, and the water was also sampled at various times upstream of and near the RO membrane. The duration of testing for each biocide was approximately 30 days. Functionality of the RO membrane, types of microbes at RO membrane after treatment, biocide efficacy on microbes in water, and biocide efficacy for biofilm growth were determined. The data is summarized in Tables 1-4.

TABLE 1

| Biocide | Flow | Rejection | P drop |
|---|---|---|---|
| None (before use) | 6.79 to 9.18 gal./min. | 99.4% to 99.7% | less than 15 psi |
| | (25.7 to 34.8 L/min.) | | (less than 0.103 MPa) |
| Bleach¹ | 5.02 gal./min. | 99.5% | 2 psi |
| | (19.0 L/min.) | | (0.014 MPa) |
| BrCl/sulfamic acid | 7.06 gal./min. | 99.6% | 3 psi |
| | (26.7 L/min.) | | (0.021 MPa) |
| Br₂DMH | 6.52 gal./min. | 99.7% | 4 psi |
| | (24.7 L/min.) | | (0.027 MPa) |

¹Comparative.

For each biocide, the RO membrane was tested after 30 days of exposure to the biocide/reducing agent system. The data in Table 1 are from membrane autopsies, and indicate whether the biocide, or at least the residual amount that contacts the RO membrane, negatively affects the membrane. The data in Table 1 show that bleach had the most negative effect on the RO membrane.

TABLE 2

| | | BrCl/ | |
|---|---|---|---|
| Biocide | Bleach¹ | sulfamic acid | Br₂DMH |
| Aerobic bacteria | Moderate | Low | High |
| Yeast/mold | Low | Low | Low |
| Rating | Low | Moderate | High |
| Aerobic bacteria | 0 to 100 | 100 to 10,000 | 10,000 to 1,000,000 |
| Yeast/mold | 0 to 100 | 100 to 10,000 | 10,000 to 100,000 |

¹Comparative.

The data in Table 2 was generated from the RO membrane as part of a membrane autopsy after 30 days of exposure to the biocide. Table 2 shows that BrCl/sulfamic acid was the most effective biocide against both aerobic bacteria and yeast/mold.

TABLE 3

| | | BrCl/ | |
|---|---|---|---|
| Biocide | Bleach¹ | sulfamic acid | Br₂DMH |
| Untreated water | 4.5 | 4.0 | 4.3 |
| (log cfu/mL) | | | |
| UF feed water | 3.6 | 3.4 | 3.1 |
| (log cfu/mL) | | | |
| RO feed water | 3.4 | 2.2 | 2.2 |
| (log cfu/mL) | | | |
| Log reduction, total | 1.1 | 1.8 | 2.1 |
| (untreated to RO) | | | |
| Reduction, total | 92.1% | 98.4% | 99.2% |
| (untreated to RO) | | | |

¹Comparative.

For the data in Table 3, the water was sampled upstream of and near the UF membrane, and upstream of and near the RO membrane The data in Table 3 are an average of samples from several days for planktonic biocidal reduction.

TABLE 4

| | Polyamide | Stainless |
|---|---|---|
| Biocide | coupon | steel coupon |
| Bleach¹ | 7480 | 4266 |
| BrCl/ | 1285 | 2158 |
| sulfamic acid | | |
| Br₂DMH | 5825 | not measured |

¹Comparative.

For the data in Table 4, the water was sampled upstream of and near the RO membrane, and a modified Robbins device was used to conduct a biofilm coupon test. The numbers in the table are RLU units, which measure the amount of ATP present on the coupon. Higher RLU values indicate that larger amounts of bacteria are present. Each value in the table is an average of 3 or 4 coupons. The data in Table 4 indicate that BrCl/sulfamic acid is the best-performing biocide against biofilm.

### Example 2

Experiments were conducted and data collected that illustrates the progression of membrane degradation tested using standard membrane testing procedures with static membrane soak tests, the membrane being in direct contact with RO-1 Biocide versus Bleach solutions in a jar over several days at multiple concentrations.

The testing was done on a Test Bench with 6 cells arranged in two parallel sets of 3 cells using components purchase. Temperatures of testing remained between 18-22 deg. C. which would have minimal impact on the estimation of membrane salt rejection. Rejection was estimated using a Conductivity Meter (Hach) to measure Permeate and Feed conductivity. See Table 5.

TABLE 5
Membrane Test Conditions

| Parameter | Value | |
|---|---|---|
| Salt Concentration | 2000 | ppm |
| Test Pressure | 300 | psi |
| Crossflow Rate | 1 | gpm |
| Duration of Test Time | 15 | minutes |
| to Equilibrate (typical) | | |
| after Mounting Membranes | | |
| in Cells | | |

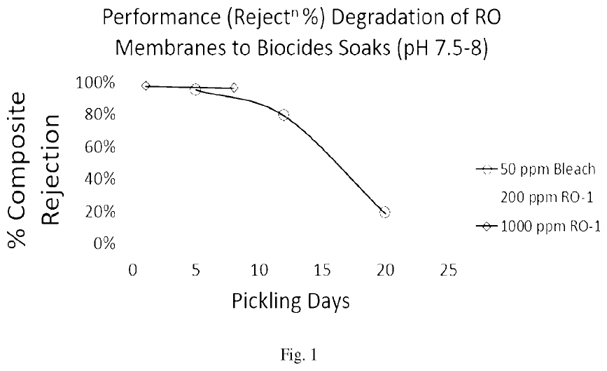

The data illustrates that at concentrations of 50 ppm, membrane rapidly deteriorates from a near perfect retention of salt concentration in the solution summarized in the influent feed parameters to about 20% after 20 days of exposure, where as the degradation rate with RO-1 appears to be relatively much more gradual up until 8 days of exposure at substantively higher concentrations of 200 ppm and 1,000 ppm. See Table 6 and FIG. 1.

TABLE 6

| | | | | 200 | 1000 | |
|---|---|---|---|---|---|---|
| | | | | ppm | ppm | |
| | 50 ppm Bleach | | | RO-1 | RO-1 | |
| Period—Days | 5 | 12 | 20 | 8 | 1 | 8 |

Averaged Test Parameters

| | | | | | | |
|---|---|---|---|---|---|---|
| Temperature (C.) | 21.2 | 20.9 | 20.8 | 20.7 | 20.2 | 18.8 |
| Collection Time | 18.0 | 20.0 | 10.0 | 25.0 | 20.0 | 10.0 |
| (min) | | | | | | |
| Permeate | 183.3 | 269.3 | 271.5 | 102.5 | 88.3 | 34.9 |
| Mass (g) | | | | | | |
| Permeat Flow | 10.2 | 13.5 | 27.2 | 4.1 | 4.4 | 3.5 |
| (g/min) | | | | | | |
| Conductivity | 185.9 | 782.0 | 2944.0 | 22.7 | 85.2 | 122.1 |
| (μs/cm) | | | | | | |
| Composite | 95.03% | 79.03% | 18.90% | 99.38% | 97.70% | 96.64% |
| Rejection | | | | | | |
| Total Membrane | 6 | 6 | 6 | 6 | 6 | 6 |
| Coupons Tested | | | | | | |

Testing with membrane coupons soaked in RO-1 solution at 1,000 ppm over 20+ days is, even at such a very high concentration, expected to remain higher performing than that exposed to 50 ppm bleach over the same duration. The concentrations selected for RO-1 are in no way indicative of the level of the biocide that will be applied for biocidal control of membranes in biofouling environments, but rather a choice for accelerated degradation studies to quickly compare the degree to which membranes are impacted by its direct contact.

Further embodiments of the invention include, without limitation:

A) A process for controlling biofouling in an aqueous membrane separation system comprising water and one or more protected membranes, which process comprises:
I) contacting, upstream of the protected membrane(s), the feed water and a biocidal amount of a bromine-containing biocide to form treated water;
II) measuring a bromine residual in the treated water at a monitoring location which is downstream of the contacting in I) and upstream of the protected membrane(s); and
III) contacting the treated water and a reducing amount of one or more reducing agents near or downstream of the monitoring location and upstream of the protected membrane(s);
   wherein the reducing agent is capable of reducing biocidal bromine to bromide ions; and
   wherein the bromine-containing biocide comprises:
a) one or more 1,3-dibromo-5,5-dialkylhydantoins;
b) one or more N,N′-bromochloro-5,5-dialkylhydantoins;
c) one or more alkali metal hypobromites and/or one or more alkaline earth metal hypobromites, optionally formed in water from one or more bromide sources and one or more hypochlorite salts and/or hypochlorous acid;
d) a bromine-based biocide formed in water from (i) one or more bromide sources, (ii) an oxidant, optionally (iii) at least one inorganic base, and optionally (iv) sulfamic acid and/or a metal salt of sulfamic acid;
e) a bromine-based biocide formed in water from (i) bromine chloride or bromine chloride and bromine, with or without conjoint use of chlorine, and (ii) overbased alkali metal salt of sulfamic acid and/or sulfamic acid, alkali metal base, and water, wherein (i) and (ii) are in relative proportions such that there is an atom ratio of nitrogen to active bromine greater than 0.93, and wherein the bromine-based biocide has a pH of greater than 7;
f) a bromine-based biocide formed in water by ozonation of one or more bromide sources; or
g) a bromine-based biocide formed in water electrolytically from one or more bromide sources.

B) A process as in A) wherein the bromine-containing biocide comprises one or more N,N′-bromochloro-5,5-dialkylhydantoins, one or more 1,3-dibromo-5,5-dialkylhydantoins, or a bromine-based biocide of e).

C) A process as in A) wherein the bromine-containing biocide comprises N,N′-bromochloro-5,5-dimethylhydantoin, 1,3-dibromo-5,5-dimethylhydantoin, or a bromine-based biocide of e) formed in water from bromine chloride.

D) A process as in B) or C) wherein the bromine-containing biocide comprises a bromine-based biocide formed in water from ingredients in bromine-based biocide e), and wherein:
said alkali metal base of (ii) is sodium hydroxide;
said biocide has an active bromine content is about 100,000 ppm or more; and/or
said pH is about 10 or greater.

E) A process as in any of A)-C) wherein the 1,3-dibromo-5,5-dialkylhydantoin(s) and/or N,N′-bromochloro-5,5-dialkylhydantoin(s) is pre-mixed with water prior to contact with the feed water, and optionally wherein, in the pre-mix water, the 1,3-dibromo-5,5-dialkylhydantoin(s) and/or N,N′-bromochloro-5,5-dialkylhydantoin(s) is in an amount sufficient to provide a bromine residual in a range of about 300 ppm to about 3500 ppm (wt/wt) as free bromine.

F) A process as in D) wherein said bromine-based biocide of e) is diluted to form a diluted bromine-based biocide of e) before contacting the feed water.

G) A process as in A) wherein the bromine-containing biocide comprises a bromine-based biocide of f); or a bromine-based biocide of g).

H) A process as in A) wherein the biocide is an N,N′-bromochloro-5,5-dialkylhydantoin or a 1,3-dibromo-5,5-dialkylhydantoin, and wherein the contacting with the feed water is performed with the biocide dissolved in water.

I) A process as in H) wherein the biocide is N,N′-bromochloro-5,5-dimethylhydantoin or 1,3-dibromo-5,5-dimethylhydantoin.

J) A process as in A) wherein the biocide is a bromine-based biocide formed in water from bromine chloride or bromine chloride and bromine, and wherein the contacting of the bromine-based biocide with the feed water is performed without dilution of the biocide.

K) A process as in J) wherein the bromine-based biocide is formed in water from bromine chloride.

L) A process as in A) wherein the one or more bromide sources for biocide c) is an alkali metal bromide and/or wherein the one or more hypochlorite salts and/or hypochlorous acid for biocide c) is an alkali metal hypochlorite.

M) A process as in L) wherein the bromide source is an alkali metal bromide and/or wherein the chlorine source is a hypochlorite.

N) A process as in L) wherein the bromide source is an sodium bromide and/or wherein the chlorine source is sodium hypochlorite.

O) A process as in A) wherein in biocide d)
the oxidant is a chlorine oxidant or an oxygen-based oxidant, and an inorganic base is present;
the oxidant is a chlorine oxidant or an oxygen-based oxidant, and sulfamic acid and/or a metal salt of sulfamic acid is present; or
the oxidant is a chlorine oxidant or an oxygen-based oxidant, and an inorganic base and sulfamic acid and/or a metal salt of sulfamic acid are present.

P) A process as in any of A)-O) wherein said bromine-containing biocide provides a bromine residual in the range of about 0.2 ppm to about 20 ppm (wt/wt) as free bromine in the treated water.

Q) A process as in any of A)-O) wherein said bromine-containing biocide provides a bromine residual in the range of about 0.5 ppm to about 10 ppm (wt/wt) as free bromine in the treated water.

R) A process as in any of A)-Q) wherein the measuring is by a chemical method or by a spectroscopic method.

S) A process as in R) wherein the measuring is a measuring of the potential of the treated water.

T) A process as in S) wherein the measuring is performed by an oxidation-reduction meter.

U) A process as in S) wherein the measuring is performed by an amperometer.

V) A process as in any of A)-U) wherein the aqueous membrane separation system is a reverse osmosis system.

Components referred to by chemical name or formula anywhere in the specification or claims hereof, whether referred to in the singular or plural, are identified as they exist prior to coming into contact with another substance referred to by chemical name or chemical type (e.g., another component, a solvent, or etc.). It matters not what chemical changes, transformations and/or reactions, if any, take place in the resulting mixture or solution as such changes, transformations, and/or reactions are the natural result of bringing the specified components together under the conditions called for pursuant to this disclosure. Thus the components are identified as ingredients to be brought together in connection with performing a desired operation or in forming a desired composition. Also, even though the claims hereinafter may refer to substances, components and/or ingredients in the present tense (“comprises”, “is”, etc.), the reference is to the substance, component or ingredient as it existed at the time just before it was first contacted, blended or mixed with one or more other substances, components and/or ingredients in accordance with the present disclosure. The fact that a substance, component or ingredient may have lost its original identity through a chemical reaction or transformation during the course of contacting, blending or mixing operations, if conducted in accordance with this disclosure and with ordinary skill of a chemist, is thus of no practical concern.

The invention may comprise, consist, or consist essentially of the materials and/or procedures recited herein.

As used herein, the term “about” modifying the quantity of an ingredient in the compositions of the invention or employed in the methods of the invention refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term about also encompasses amounts that differ due to different equilibrium conditions for a composition resulting from a particular initial mixture. Whether or not modified by the term “about”, the claims include equivalents to the quantities.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

This invention is susceptible to considerable variation in its practice. Therefore the foregoing description is not intended to limit, and should not be construed as limiting, the invention to the particular exemplifications presented hereinabove.

1. A process for controlling biofouling in an aqueous membrane separation system comprising water and one or more protected membranes, which process comprises:
I) contacting, upstream of the protected membrane(s), feed water and a biocidal amount of a bromine-containing biocide to form treated water;
II) measuring a bromine residual in the treated water at a monitoring location which is downstream of the contacting in I) and upstream of the protected membrane(s); and
III) contacting the treated water and a reducing amount of one or more reducing agents near or downstream of the monitoring location and upstream of the protected membrane(s);
wherein the reducing agent is capable of reducing biocidal bromine to bromide ions; and
wherein the bromine-containing biocide comprises:
A) one or more 1,3-dibromo-5,5-dialkylhydantoins;
B) one or more N,N′-bromochloro-5,5-dialkylhydantoins;
C) one or more alkali metal hypobromites and/or one or more alkaline earth metal hypobromites, formed in water from (i) one or more bromide sources and (ii) one or more hypochlorite salts and/or hypochlorous acid;
D) a bromine-based biocide formed in water from (i) one or more bromide sources, (ii) an oxidant, (iii) at least one inorganic base, and (iv) sulfamic acid and/or a metal salt of sulfamic acid;
E) a bromine-based biocide formed in water from (i) bromine chloride or bromine chloride and bromine, with or without conjoint use of chlorine, and (ii) overbased alkali metal salt of sulfamic acid and/or sulfamic acid, alkali metal base, and water, wherein (i) and (ii) are in relative proportions such that there is an atom ratio of nitrogen to active bromine greater than 0.93, and wherein the bromine-based biocide has a pH of greater than 7;
F) a bromine-based biocide formed in water by ozonation of one or more bromide sources; or
G) a bromine-based biocide formed in water electrolytically from one or more bromide sources.

2. A process as in claim 1 wherein the bromine-containing biocide comprises one or more N,N′-bromochloro-5,5-dialkylhydantoins, one or more 1,3-dibromo-5,5-dialkylhydantoins, or a bromine-based biocide of E).

3. A process as in claim 1 wherein the bromine-containing biocide comprises N,N′-bromochloro-5,5-dimethylhydantoin, 1,3-dibromo-5,5-dimethylhydantoin, or a bromine-based biocide of E) formed in water from bromine chloride.

4. A process as in claim 2 [2 or 3] wherein the bromine-containing biocide comprises a bromine-based biocide formed in water from ingredients in bromine-based biocide E), and wherein:
said alkali metal base of (ii) is sodium hydroxide;
said biocide has an active bromine content is about 100,000 ppm or more; and/or
said pH is about 10 or greater.

5. A process as in claim 1 wherein the 1,3-dibromo-5,5-dialkylhydantoin(s) and/or N,N′-bromochloro-5,5-dialkylhydantoin(s) is pre-mixed with water prior to contact with the feed water, and wherein, in the pre-mix water, the 1,3-dibromo-5,5-dialkylhydantoin(s) and/or N,N′-bromochloro-5,5-dialkylhydantoin(s) is in an amount sufficient to provide a bromine residual in a range of about 300 ppm to about 3500 ppm (wt/wt) as free bromine.

6. A process as in claim 4 wherein said bromine-based biocide of E) is diluted to form a diluted bromine-based biocide of E) before contacting the feed water.

7. A process as in claim 1 wherein the bromine-containing biocide comprises:
a bromine-based biocide of F); or
a bromine-based biocide of G).

8. A process as in claim 1 wherein the biocide is an N,N′-bromochloro-5,5-dialkylhydantoin or a 1,3-dibromo-5,5-dialkylhydantoin, and wherein the contacting with the feed water is performed with the biocide dissolved in water.

9. A process as in claim 8 wherein the biocide is N,N′-bromochloro-5,5-dimethylhydantoin or 1,3-dibromo-5,5-dimethylhydantoin.

10. A process as in claim 1 wherein the biocide is a bromine-based biocide formed in water from bromine chloride or bromine chloride and bromine, and wherein the contacting of the bromine-based biocide with the feed water is performed without dilution of the biocide.

11. A process as in claim 10 wherein the bromine-based biocide is formed in water from bromine chloride.

12. A process as in claim 1 wherein the one or more bromide sources for biocide C) is an alkali metal bromide and/or wherein the one or more hypochlorite salts and/or hypochlorous acid for biocide C) is an alkali metal hypochlorite.

13. A process as in claim 12 wherein the alkali metal bromide is sodium bromide and/or wherein the alkali metal hypochlorite is sodium hypochlorite.

14. A process as in claim 1 wherein in biocide D)
the oxidant is a chlorine oxidant or an oxygen-based oxidant, and an inorganic base is present; the oxidant is a chlorine oxidant or an oxygen-based oxidant, and sulfamic acid and/or a metal salt of sulfamic acid is present; or
the oxidant is a chlorine oxidant or an oxygen-based oxidant, and an inorganic base and sulfamic acid and/or a metal salt of sulfamic acid are present.

15. A process as in claim 1 wherein said bromine-containing biocide provides a bromine residual in the range of about 0.2 ppm to about 20 ppm (wt/wt) as free bromine in the treated water.

16. A process as in claim 1 wherein said bromine-containing biocide provides a bromine residual in the range of about 0.5 ppm to about 10 ppm (wt/wt) as free bromine in the treated water.

17. A process as in claim 1 wherein the measuring is by a chemical method or by a spectroscopic method.

18. A process as in claim 17 wherein the measuring is a measuring of the potential of the treated water.

19. A process as in claim 18 wherein the measuring is performed by an oxidation-reduction meter.

20. A process as in claim 18 wherein the measuring is performed by an amperometer.

21. A process as in claim 1 wherein the aqueous membrane separation system is a reverse osmosis system.
